SEQUENCE LISTING (7 sequences)

SEQ ID NO: 1 - protein, 534 aa, Candida rugosa
APTATLANGDTITGLNAIINEAFLGIPFAEPPVGNLRFKDPVPYSGSLDGQKFTSYGPSCMQQNPEGTYEENLPKAALDLVMQSKVFEAVSPSSEDCLTINVVRPPGTKAGANLPVMLWIFGGGFEVGGTSTFPPAQMITKSIAMGKPIIHVSVNYRVSSWGFLAGDEIKAEGSANAGLKDQRLGMQWVADNIAAFGGDPTKVTIFGESAGSMSVMCHILWNDGDNTYKGKPLFRAGIMQSGAMVPSDAVDGIYGNEIFDLLASNAGCGSASDKLACLRGVSSDTLEDATNNTPGFLAYSSLRLSYLPRPDGVNITDDMYALVREGKYANIPVIIGDQNDEGTFFGTSSLNVTTDAQAREYFKQSFVHASDAEIDTLMTAYPGDITQGSPFDTGILNALTPQFKRISAVLGDLGFTLARRYFLNHYTGGTKYSFLSKQLSGLPVLGTFHSNDIVFQDYLLGSGSLIYNNAFIAFATDLDPNTAGLLVKWPEYTSSSQSGNNLMMINALGLYTGKDNFRTAGYDALFSNPPSFFV

SEQ ID NO: 2 - DNA, 1602 bp, Artificial Sequence
  /organism=“Artificial Sequence”/note=“Codon optimized nucleotide sequence for expression in Pichia pastoris of the mature coding sequence of Lip1 of Candida rugosa ”/mol_type=“unassigned DNA”
  misc_feature (1)..(1602): Codon optimized nucleotide sequence for expression in Pichia pastoris of the mature coding sequence of Lip1 of Candida rugosa
gccccaaccgccactttggctaacggtgacaccatcaccggtttgaacgccatcatcaacgaagccttcttgggtattccatttgccgaaccaccagttggtaacttgagattcaaggacccagttccatactccggttccttggatggtcaaaagttcacttcttacggtccatcttgtatgcaacaaaacccagaaggtacctacgaagaaaacttgccaaaggcagctttagatctggttatgcaatccaaagttttcgaagctgtttctccatcttctgaagactgtttgaccattaatgttgttagaccacccgggacaaaggctggtgccaacttgccagttatgttgtggatctttggtggtggttttgaagttggtggtactagtaccttccctccagcccaaatgattaccaagtctattgctatgggtaagccaatcatccacgtttctgtcaactacagagtctccagctggggtttcttggctggtgacgaaatcaaggccgaaggttctgccaacgccggtttgaaggaccaaagattgggtatgcaatgggtggctgacaacattgctgcttttggtggtgatccaactaaggttactatctttggtgaatctgctggttctatgtccgtcatgtgtcacattttgtggaacgacggtgacaacacttacaagggtaagccattgttcagagctggtatcatgcaatctggtgctatggttccatctgacgccgtcgacggtatctacggtaacgaaatttttgacttgttggcttccaacgctggttgtggttctgcctctgacaagttggcttgtttgagaggtgtttcttctgacactttggaagacgccaccaacaacacccctggtttcttggcttactcctccttaagattgtcttacttgccaagaccagacggtgttaacatcaccgacgacatgtacgctttggttagagaaggtaagtatgccaacatccctgttatcatcggtgaccaaaacgacgaaggtaccttctttggtacttcttctttgaacgttaccactgatgcccaagccagagaatatttcaagcaatcttttgtccacgctagcgacgctgaaatcgacactttgatgactgcttacccaggtgacatcactcaaggttctccatttgacactggaattctaaacgccttgaccccacaattcaagagaatctctgctatcttgggtgacttgggttttactttggctcgtagatacttcttgaaccactacaccggtggtaccaagtactctttcttgtctaagcaattgtctggtttgccagttttgggtactttccactccaacgatatcgtcttccaagactacttgttgggttctggttccttgatctacaacaacgctttcattgcttttgccactgacttggacccaaacaccgccggtttgttggttaagtggccagaatacacctcttcttctcaatctggtaacaacttgatgatgatcaacgctttgggtttgtacaccggtaaggacaacttcagaaccgccggttacgacgctttgttctccaacccaccatctttctttgtt

SEQ ID NO: 3 - DNA, 2582 bp, Komagataella phaffii
  source (1)..(2582): /organism=“Komagataella phaffii” /note=“strain ATCC 76273; HIS4 gene” /mol_type=“unassigned DNA”
aatacggcttcagaatttctcaagactacactcactgtccgacttcaagtatgacatttcccttgctacctgcatacgcaagtgttgcagagtttgataattccttgagtttggtaggaaaagccgtgtttccctatgctgctgaccagctgcacaacctgatcaagttcactcaatcgactgagcttcaagttaatgtgcaagttgagtcatccgttacagaggaccaatttgaggagctgatcgacaacttgctcaagttgtacaataatggtatcaatgaagtgattttggacctagatttggcagaaagagttgtccaaaggatcccaggcgctagggttatctataggaccctggttgataaagttgcatccttgcccgctaatgctagtatcgctgtgcctttttcttctccactgggcgatttgaaaagtttcactaatggcggtagtagaactgtttatgctttttctgagaccgcaaagttggtagatgtgacttccactgttgcttctggtataatccccattattgatgctcggcaattgactactgaatacgaactttctgaagatgtcaaaaagttccctgtcagtgaaattttgttggcgtctttgactactgaccgccccgatggtctattcactactttggtggctgactcttctaattactcgttgggcctggtgtactcgtccaaaaagtctattccggaggctataaggacacaaactggagtctaccaatctcgtcgtcacggtttgtggtataaaggtgctacatctggagcaactcaaaagttgctgggtatcgaattggattgtgatggagactgcttgaaatttgtggttgaacaaacaggtgttggtttctgtcacttggaacgcacttcctgttttggccaatcaaagggtcttagagccatggaagccaccttgtgggatcgtaagagcaatgctccagaaggttcttataccaaacggttatttgacgacgaagttttgttgaacgctaaaattagggaggaagctgatgaacttgcagaagctaaatccaaggaagatatagcctgggaatgtgctgacttattttattttgcattagttagatgtgccaagtacggtgtgacgttggacgaggtggagagaaacctggatatgaagtccctaaaggtcactagaaggaaaggagatgccaagccaggatacaccaaggaacaacctaaagaagaatccaaacctaaagaagtcccttctgaaggtcgtattgaattgtgcaaaattgacgtttctaaggcctcctcacaagaaattgaagatgcccttcgtcgtcctatccagaaaacggaacagattatggaattagtcaaaccaattgtcgacaatgttcgtcaaaatggtgacaaagcccttttagaactaactgccaagtttgatggagtcgctttgaagacacctgtgttagaagctcctttcccagaggaacttatgcaattgccagataacgttaagagagccattgatctctctatagataacgtcaggaaattccatgaagctcaactaacggagacgttgcaagttgagacttgccctggtgtagtctgctctcgttttgcaagacctattgagaaagttggcctctatattcctggtggaaccgcaattctgccttccacttccctgatgctgggtgttcctgccaaagttgctggtcgcaaagaaattgtttttgcatctccacctaagaaggatggtacccttaccccagaagtcatctacgttgcccacaaggttggtgctaagtgtatcgtgctagcaggaggcgcccaggcagtagctgctatggcttacggaacagaaactgttcctaagtgtgacaaaatatttggtccaggaaaccagttcgttactgctgccaagatgatggttcaaaatgacacatcagccctgtgtagtattgacatgcctgctgggccttctgaagttctagttattgctgataaatacgctgatccagatttcgttgcctcagaccttctgtctcaagctgaacatggtattgattcccaggtgattctgttggctgtcgatatgacagacaaggagcttgccagaattgaagatgctgttcacaaccaagctgtgcagttgccaagggttgaaattgtacgcaagtgtattgcacactctacaaccctatcggttgcaacctacgagcaggctttggaaatgtccaatcagtacgctcctgaacacttgatcctgcaaatcgagaatgcttcttcttatgttgatcaagtacaacacgctggatctgtgtttgttggtgcctactctccagagagttgtggagattactcctccggtaccaaccacactttgccaacgtacggatatgcccgtcaatacagcggagttaacactgcaaccttccagaagttcatcacttcacaagacgtaactcctgagggactgaaacatattggccaagcagtgatggatctggctgctgttgaaggtctagatgctcaccgcaatgctgttaaggttcgtatggagaaactgggacttatttaa

SEQ ID NO: 4 - DNA, 34 bp, Artificial Sequence
  /organism=“Artificial Sequence” /note=“ Nucleotide sequence of an 34 bp FRT recombination site ” /mol_type=“unassigned DNA”
  misc_feature (1)..(34): Nucleotide sequence of an 34 bp FRT recombination site
gaagttcctatactttctagagaataggaacttc

SEQ ID NO: 5 - protein, 4 aa, Artificial Sequence
  Glutamine Alanine repeat
EAEA

SEQ ID NO: 6 - protein, 89 aa, Artificial Sequence
  -mating factor from S. cerevisiae followed by a Kex2 processing site (KR) and Glutamine Alanine repeat
MRFPSIFTAVLFAASSALAAPVNTTTEDETAQIPAEAVIGYSDLEGDFDVAVLPFSNSTNNGLLFINTTIASIAAKEEGVSLEKREAEA

SEQ ID NO: 7 - DNA, 1650 bp, Artificial Sequence
  /organism=“Artificial Sequence”/note=“Sequence encoding Kex2 processing, Glutamine Alanine repeat and codon optimized C. rugosa 534 wt XhoI / NotI sites
  misc_feature (1)..(1650): Sequence encoding Kex2 processing, Glutamine Alanine repeat and codon optimized C. rugosa 534 wt XhoI / NotI sites
ctcgagaagagagaggccgaagctgccccaaccgccactttggctaacggtgacaccatcaccggtttgaacgccatcatcaacgaagccttcttgggtattccatttgccgaaccaccagttggtaacttgagattcaaggacccagttccatactccggttccttggatggtcaaaagttcacttcttacggtccatcttgtatgcaacaaaacccagaaggtacctacgaagaaaacttgccaaaggcagctttagatctggttatgcaatccaaagttttcgaagctgtttctccatcttctgaagactgtttgaccattaatgttgttagaccacccgggacaaaggctggtgccaacttgccagttatgttgtggatctttggtggtggttttgaagttggtggtactagtaccttccctccagcccaaatgattaccaagtctattgctatgggtaagccaatcatccacgtttctgtcaactacagagtctccagctggggtttcttggctggtgacgaaatcaaggccgaaggttctgccaacgccggtttgaaggaccaaagattgggtatgcaatgggtggctgacaacattgctgcttttggtggtgatccaactaaggttactatctttggtgaatctgctggttctatgtccgtcatgtgtcacattttgtggaacgacggtgacaacacttacaagggtaagccattgttcagagctggtatcatgcaatctggtgctatggttccatctgacgccgtcgacggtatctacggtaacgaaatttttgacttgttggcttccaacgctggttgtggttctgcctctgacaagttggcttgtttgagaggtgtttcttctgacactttggaagacgccaccaacaacacccctggtttcttggcttactcctccttaagattgtcttacttgccaagaccagacggtgttaacatcaccgacgacatgtacgctttggttagagaaggtaagtatgccaacatccctgttatcatcggtgaccaaaacgacgaaggtaccttctttggtacttcttctttgaacgttaccactgatgcccaagccagagaatatttcaagcaatcttttgtccacgctagcgacgctgaaatcgacactttgatgactgcttacccaggtgacatcactcaaggttctccatttgacactggaattctaaacgccttgaccccacaattcaagagaatctctgctatcttgggtgacttgggttttactttggctcgtagatacttcttgaaccactacaccggtggtaccaagtactctttcttgtctaagcaattgtctggtttgccagttttgggtactttccactccaacgatatcgtcttccaagactacttgttgggttctggttccttgatctacaacaacgctttcattgcttttgccactgacttggacccaaacaccgccggtttgttggttaagtggccagaatacacctcttcttctcaatctggtaacaacttgatgatgatcaacgctttgggtttgtacaccggtaaggacaacttcagaaccgccggttacgacgctttgttctccaacccaccatctttctttgtttaataaggttaaaggggcggccgc

(19) United States
(12) Patent Application Publication
(10) Pub. No.: US 2023/0250406 A1
(43) Pub. Date: Aug. 10, 2023

(54) MUTANT LIPASE AND USE THEREOF

(71) Applicant: DSM IP Assets B.V., Heerlen (NL)

(72) Inventors: René Marcel DE JONG, Echt (NL); Willem BIJLEVELD, Echt (NL); Paul Frederick MUGFORD, Novia Scotia (CA)

(21) Appl. No.: 17/056,230

(22) PCT Filed: May 17, 2019
(86) PCT No.: PCT/EP2019/062787

§ 371 Date: Nov. 17, 2020

(30) Foreign Application Priority Data
May 18, 2018 (EP) 18173104.3

(51) Int. Cl. C12N 9/20; A23D 9/04

(52) U.S. Cl. CPC C12N 9/20; C12Y 301/01003; A23D 9/04

(57) ABSTRACT

The present invention relates to a polypeptide having lipase activity wherein the polypeptide when aligned with the polypeptide according to SEQ ID NO: 1, comprises at least an amino acid substitution G414X, wherein the numbering of amino acid position(s) is/are defined with reference to SEQ ID NO: 1. The invention further relates to a process for preparing a product comprising an oil or fat comprising bringing an intermediary form of the product comprising oil or fat into contact with a polypeptide as disclosed herein and the use of a polypeptide as disclosed herein to saturated fatty acids in an oil or fat.

The present invention relates to a recombinant polypeptide having lipase activity, a composition comprising the polypeptide, a nucleic acid encoding a polypeptide having a lipase activity, an expression vector comprising the nucleic acid encoding a polypeptide having a lipase activity, a recombinant host cell comprising the expression vector, a method for preparing a recombinant polypeptide having lipase activity and a process for preparing a food or feed product wherein the lipase is used.

### BACKGROUND

Fish oil is a valuable source of long chain (LC) polyunsaturated omega-3 fatty acids, in particular eicosapentaenoic acid (EPA; C20:5) and docosahexaenoic acid (DHA; C22:6). These LC omega-3 fatty acids have shown to contribute to a healthy lifestyle and the human consumption of fish oil has shown an increase the last decades. However, fish oil not only contains healthy LC-omega-3 fatty acids. Part of the fish oil consists of less healthy saturated fatty acids such as palmitic acid (C16:0). Accordingly, various methods have been developed to increase the concentration of EPA and DHA relative to palmitic acid.

Similarly, soy oil is a valuable source of linoleic and oleic acid. However, soy oil also contains less healthy saturated fatty acids such as palmitic acid.

US2016/0229785 for instance discloses a continuous process for direct extraction of an omega-3 fatty acids enriched triglyceride product from a crude fish oil, wherein the fish oil is mixed with a solvent and passing to a polar phase simulated moving bed adsorption zone. A disadvantage of this process is that a solvent is applied in the extraction process.

CN105349587A discloses a method for improving contents of EPA and DHA in glyceride type of fish oil, by contacting a freeze-dried strain of Aspergillus oryzae with a fish oil and ethyl ester fish oil as substrates, wherein an ester interchange is catalyzed by an Aspergillus oryzae lipase.

Alternatively lipases are used to increase the concentration of EPA and DHA in fish oil. Fernandez-Lorent et. al. (2011) J. Am Oil Chem Soc 88: 1173-1178 discloses the influence of different hydrophobic supports for immobilizing lipases on the release of omega-3 fatty acids by the lipases.

Lipases (triacylglycerol acyl hydrolase, EC 3.1.1.3) are part of the family of hydrolases that act on carboxylic acid. Lipases can be produced by various microorganisms. Candida rugosa lipases are widely used in industry and five different lipase amino acid sequences have been identified. Schmitt et al. (2002), Protein Engineering, Vol. 15, no. 7, pp 595-601 discloses several Candida rugosa lipase mutants with different substrate specificity.

The present invention relates to a lipase that can lower the content of saturated fatty acids, such as palmitic acid in a product.

### SUMMARY

Disclosed herein is a polypeptide having lipase activity wherein the polypeptide is selected from the group consisting of
a) a polypeptide, which, when aligned with the polypeptide according to SEQ ID NO: 1, comprises at least the amino acid substitution G414X, wherein the numbering of amino acid position(s) is/are defined with reference to SEQ ID NO: 1, or corresponding position;
b) a polypeptide according to a), wherein the polypeptide has at least 80%, 85%, 90%, 91%, 92%, 93%, 94%, 95%, 96%, 97%, 98%% or 99% identity to the amino sequence of SEQ ID NO: 1;
c) a polypeptide encoded by a nucleic acid which has at least 80%, 85%, 90%, 91%, 92%, 93%, 94%, 95%, 96%, 97%, 98%, or 99% identity to the nucleotide sequence of SEQ ID NO: 2, wherein SEQ ID NO: 2 comprises at least one mutation resulting in at least the amino acid substitution G414X of a polypeptide according to SEQ ID NO: 1 or corresponding position, wherein the numbering of amino acid position(s) is/are defined with reference to SEQ ID NO: 1; and,
d) a polypeptide encoded by a nucleic acid comprising a sequence that hybridizes under low, medium and/or high stringency conditions to the complementary strand of sequence of SEQ ID NO: 2, wherein SEQ ID NO: 2 comprises at least one mutation resulting in at least the amino acid substitution G414X of a polypeptide according to SEQ ID NO: 1 or corresponding position, wherein the numbering of amino acid position(s) is/are defined with reference to SEQ ID NO: 1.

Surprisingly, it was found that the ratio of lipase activity on palmitic acid relative to the lipase activity on eicosapentaenoic acid (EPA), linoleic acid and oleic acid of a polypeptide as disclosed herein was higher than this ratio of a corresponding wild type polypeptide. Preferably, the ratio of the lipase activity on palmitic acid relative to the lipase activity on eicosapentaenoic acid (EPA), linoleic acid and/or oleic acid of a polypeptide as disclosed herein is between 1.5 to 2, 1.5 to 3, 1.5 to 4, 1.5 to 5, 1.5 to 6, 1.5 to 7, 1.5 to 8, 1.5 to 9, 1.5 to 10, 1.5 to 20 or between 5 and 100 times higher than this ratio of a corresponding wild type polypeptide, for instance a polypeptide comprising SEQ ID NO: 1.

In another aspect the present invention provides a method of generating a variant polypeptide having lipase activity as disclosed herein.

The invention also provides a nucleic acid encoding a lipase, wherein the nucleic acid has at least 80%, 85%, 90%, 95%, 96%, 97%, 98%, or 99% sequence identity to SEQ ID NO: 2, wherein SEQ ID NO: 2 comprises at least one mutation resulting in the amino acid substitution G414X, and optionally one or more amino acid substitutions chosen from the group consisting of I100V, S450A, L413M, L410F, S365A, S365Q, Y361W, F362L, and V409A, of a polypeptide according to SEQ ID NO: 1, wherein the numbering of amino acid position(s) is/are defined with reference to SEQ ID NO: 1, or corresponding position.

In another aspect the present invention relates to an expression vector comprising a nucleic acid encoding a polypeptide as disclosed herein.

In another aspect the present invention relates to a recombinant host cell comprising a nucleic acid, or an expression vector as disclosed herein.

In yet another aspect the present invention relates to a method for the preparation of a polypeptide, comprising cultivating a host cell as disclosed herein under conditions that allow expression of the polypeptide, and preparing the polypeptide.

In one aspect the present invention relates to a process for preparing a product comprising an oil or fat comprising bringing the oil or fat into contact with a polypeptide as disclosed herein.

In another aspect the present invention relates to the use of a polypeptide as disclosed herein to lower the concentration of palmitic acid in a fat or oil.

### Definitions

The term “complementary strand” can be used interchangeably with the term “complement”. The complement of a nucleic acid strand can be the complement of a coding strand or the complement of a non-coding strand. When referring to double-stranded nucleic acids, the complement of a nucleic acid encoding a polypeptide refers to the complementary strand of the strand encoding the amino acid sequence or to any nucleic acid molecule containing the same.

The term “control sequence” can be used interchangeably with the term “expression-regulating nucleic acid sequence”. The term as used herein refers to nucleic acid sequences necessary for and/or affecting the expression of an operably linked coding sequence in a particular host organism or in vitro. When two nucleic acid sequences are operably linked, they usually will be in the same orientation and also in the same reading frame. They usually will be essentially contiguous, although this may not be required. The expression-regulating nucleic acid sequences, such as inter alia appropriate transcription initiation, termination, promoter, leader, signal peptide, propeptide, prepropeptide, or enhancer sequences; Shine-Dalgarno sequence, repressor or activator sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion, can be any nucleic acid sequence showing activity in the host organism of choice and can be derived from genes encoding proteins, which are either endogenous or heterologous to a host cell. Each control sequence may be native or foreign to the nucleic acid sequence encoding the polypeptide. When desired, the control sequence may be provided with linkers for the purpose of introducing specific restriction sites facilitating ligation of the control sequences with the coding region of the nucleic acid sequence encoding a polypeptide. Control sequences may be optimized to their specific purpose.

The term “expression” includes any step involved in the production of the polypeptide including, but not limited to, transcription, post transcriptional modification, translation, post-translational modification, and secretion.

Nucleic acids of the present invention as described herein may be over-expressed in a host cell of the invention compared to a parent cell in which said gene is not over-expressed. Over-expression of a polynucleotide sequence is defined herein as the expression of said sequence gene which results in an activity of the polypeptide encoded by the said sequence in a host cell being at least 1.1, at least 1.25 or at least 1.5-fold the activity of the polypeptide in the host cell; preferably the activity of said polypeptide is at least 2-fold, more preferably at least 3-fold, more preferably at least 4-fold, more preferably at least 5-fold, even more preferably at least 10-fold and most preferably at least 20-fold the activity of the polypeptide in the parent cell.

An “expression vector” comprises a polynucleotide coding for a polypeptide, such as a polypeptide according to the present invention, operably linked to the appropriate control sequences (such as a promoter, and transcriptional and translational stop signals) for expression and/or translation in vitro, or in a host cell of the polynucleotide. The expression vector may be any vector (e.g., a plasmid or virus), which can be conveniently subjected to recombinant DNA procedures and can bring about the expression of the polynucleotide. The choice of the vector will typically depend on the compatibility of the vector with the cell into which the vector is to be introduced. The vectors may be linear or closed circular plasmids. The vector may be an autonomously replicating vector, i.e., a vector, which exists as an extra-chromosomal entity, the replication of which is independent of chromosomal replication, e.g., a plasmid, an extra-chromosomal element, a mini-chromosome, or an artificial chromosome. Alternatively, the vector may be one which, when introduced into the host cell, is integrated into the genome and replicated together with the chromosome(s) into which it has been integrated. The integrative cloning vector may integrate at random or at a predetermined target locus in the chromosomes of the host cell. The vector system may be a single vector or plasmid or two or more vectors or plasmids, which together contain the total DNA to be introduced into the genome of the host cell, or a transposon. A vector of the invention may comprise one, two or more, for example three, four or five polynucleotides of the invention, for example for overexpression.

The term “gene” as used herein refers to a segment of a nucleic acid molecule coding for a polypeptide chain, that may or may not include gene regulatory sequences preceding and following the coding sequence, e.g. promoters, enhancers, etc., as well as intervening sequences (introns) between individual coding segments (exons). It will further be appreciated that the definition of gene can include nucleic acids that do not encode polypeptide, but rather provide templates for transcription of functional RNA molecules such as tRNAs, rRNAs, etc.

A host cell as defined herein is an organism suitable for genetic manipulation and one which may be cultured at cell densities useful for industrial production of a target product, such as a polypeptide according to the present invention. A host cell may be a host cell found in nature or a host cell derived from a parent host cell after genetic manipulation or classical mutagenesis. Advantageously, a host cell is a recombinant host cell. A host cell may be a prokaryotic, archaebacterial or eukaryotic host cell. A prokaryotic host cell may be, but is not limited to, a bacterial host cell. A eukaryotic host cell may be, but is not limited to, a yeast, a fungus, an amoeba, an algae, a plant, an animal, or an insect host cell.

The term “heterologous” as used herein refers to nucleic acid or amino acid sequences not naturally occurring in a host cell. In other words, the nucleic acid or amino acid sequence is not identical to that naturally found in the host cell.

The term “hybridization” means the pairing of substantially complementary strands of oligomeric compounds, such as nucleic acid compounds. Hybridization may be performed under low, medium or high stringency conditions. Low stringency hybridization conditions comprise hybridizing in 6× sodium chloride/sodium citrate (SSC) at about 45° C., followed by two washes in 0.2×SSC, 0.1% SDS at least at 50° C. (the temperature of the washes can be increased to 55° C. for low stringency conditions). Medium stringency hybridization conditions comprise hybridizing in 6×SSC at about 45° C., followed by one or more washes in 0.2×SSC, 0.1% SDS at 60° C., and high stringency hybridization conditions comprise hybridizing in 6×SSC at about 45° C., followed by one or more washes in 0.2×SSC, 0.1% SDS at 65° C.

An “isolated nucleic acid fragment” is a nucleic acid fragment that is not naturally occurring as a fragment and would not be found in the natural state.

The term “isolated polypeptide” as used herein means a polypeptide that is removed from at least one component, e.g. other polypeptide material, with which it is naturally associated. The isolated polypeptide may be free of any other impurities. The isolated polypeptide may be at least 50% pure, e.g., at least 60% pure, at least 70% pure, at least 75% pure, at least 80% pure, at least 85% pure, at least 80% pure, at least 90% pure, or at least 95% pure, 96%, 97%, 98%, 99%, 99.5%, 99.9% as determined by SDS-PAGE or any other analytical method suitable for this purpose and known to the person skilled in the art. An isolated polypeptide may be produced by a recombinant host cell.

A nucleic acid or polynucleotide sequence is defined herein as a nucleotide polymer comprising at least 5 nucleotide or nucleic acid units. A nucleotide or nucleic acid refers to RNA and DNA. The terms “nucleic acid” and “polynucleotide sequence” are used interchangeably herein.

A nucleic acid molecule which is complementary to another nucleotide sequence is one which is sufficiently complementary to the other nucleotide sequence such that it can hybridize to the other nucleotide sequence thereby forming a stable duplex. The term “cDNA” (complementary DNA) is defined herein as a DNA molecule which can be prepared by reverse transcription from a mRNA molecule. In prokaryotes the mRNA molecule is obtained from the transcription of the genomic DNA of a gene present in a cell. In eukaryotic cells genes contain both exons, i.e. coding sequences, and introns, i.e. intervening sequences located between the exons. Therefore in eukaryotic cells the initial, primary RNA obtained from transcription of the genomic DNA of a gene is processed through a series of steps before appearing as mRNA. These steps include the removal of intron sequences by a process called splicing. cDNA derived from mRNA only contains coding sequences and can be directly translated into the corresponding polypeptide product.

A “peptide” refers to a short chain of amino acid residues linked by a peptide (amide) bonds. The shortest peptide, a dipeptide, consists of 2 amino acids joined by single peptide bond.

The term “polypeptide” refers to a molecule comprising amino acid residues linked by peptide bonds and containing more than five amino acid residues. The term “protein” as used herein is synonymous with the term “polypeptide” and may also refer to two or more polypeptides. Thus, the terms “protein” and “polypeptide” can be used interchangeably. Polypeptides may optionally be modified (e.g., glycosylated, phosphorylated, acylated, farnesylated, prenylated, sulfonated, and the like) to add functionality. Polypeptides exhibiting activity in the presence of a specific substrate under certain conditions may be referred to as enzymes. It will be understood that, as a result of the degeneracy of the genetic code, a multitude of nucleotide sequences encoding a given polypeptide may be produced.

The term “recombinant” when used in reference to a cell, nucleic acid, protein or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, underexpressed or not expressed at all. The term “recombinant” is synonymous with “genetically modified” and “transgenic”.

“Sequence identity”, or sequence homology are used interchangeable herein. For the purpose of this invention, it is defined here that in order to determine the percentage of sequence homology or sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/bases or amino acids. The sequence identity is the percentage of identical matches between the two sequences over the reported aligned region. The percent sequence identity between two amino acid sequences or between two nucleotide sequences may be determined using the Needleman and Wunsch algorithm for the alignment of two sequences. (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, P. Longden, I. and Bleasby, A. Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: Number of corresponding positions in the alignment showing an identical amino acid or identical nucleotide in both sequences divided by the total length of the alignment after subtraction of the total number of gaps in the alignment. The identity as defined herein can be obtained from NEEDLE by using the NOBRIEF option and is labeled in the output of the program as “longest-identity”.

The nucleic acid and protein sequences disclosed herein can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., XBLAST and NBLAST) can be used. See the homepage of the National Center for Biotechnology Information at http://www.ncbi.nlm.nih.gov/.

The term “substantially pure” with regard to polypeptides refers to a polypeptide preparation which contains at the most 50% by weight of other polypeptide material. The polypeptides disclosed herein are preferably in a substantially pure form. In particular, it is preferred that the polypeptides disclosed herein are in “essentially pure form”, i.e. that the polypeptide preparation is essentially free of other polypeptide material. Optionally, the polypeptide may also be essentially free of non-polypeptide material such as nucleic acids, lipids, media components, and the like. Herein, the term “substantially pure polypeptide” is synonymous with the terms “isolated polypeptide” and “polypeptide in isolated form”.

A “substitution” as used herein in relation to polypeptides or nucleic acids, denotes the replacement of one or more amino acids in a polypeptide sequence or of one or more nucleotides in a polynucleotide sequence, respectively, by different amino acids or nucleotides, respectively. For instance, a substitution indicates that a position in a polypeptide as disclosed herein, such as a variant polypeptide, which corresponds to at least one position set out above in SEQ ID NO: 1, comprises an amino acid residue which does not appear at that position in the parent polypeptide (for instance the parent sequence SEQ ID NO: 1).

A “synthetic molecule”, such as a synthetic nucleic acid or a synthetic polypeptide is produced by in vitro chemical or enzymatic synthesis. It includes, but is not limited to, variant nucleic acids made with optimal codon usage for host organisms of choice.

A synthetic nucleic acid may be optimized for codon use, preferably according to the methods described in WO2006/077258 and/or WO2008000632, which are herein incorporated by reference. WO2008/000632 addresses codon-pair optimization. Codon-pair optimization is a method wherein the nucleotide sequences encoding a polypeptide that have been modified with respect to their codon-usage, in particular the codon-pairs that are used, are optimized to obtain improved expression of the nucleotide sequence encoding the polypeptide and/or improved production of the encoded polypeptide. Codon pairs are defined as a set of two subsequent triplets (codons) in a coding sequence. Those skilled in the art will know that the codon usage needs to be adapted depending on the host species, possibly resulting in variants with significant homology deviation from SEQ ID NO: 2, but still encoding the polypeptide according to the invention.

As used herein, the terms “variant”, “derivative”, “mutant” or “homologue” can be used interchangeably. They can refer to either polypeptides or nucleic acids. Variants include substitutions, insertions, deletions, truncations, transversions, and/or inversions, at one or more locations relative to a reference sequence. Variants can be made for example by site-saturation mutagenesis, scanning mutagenesis, insertional mutagenesis, random mutagenesis, site-directed mutagenesis, and directed-evolution, as well as various other recombination approaches known to a skilled person in the art. Variant genes of nucleic acids may be synthesized artificially by known techniques in the art.

### FIGURES

FIG. 1. Physical map of the integration expression vector, pD902-LIP1. The XhoI and NotI sites were used to introduce the lip1 lipase gene. The digestion with SacI targets the integration to the AOX1 site in Pichia pastoris. Transformants were selected on zeocin.

FIG. 2 shows the extent of palmitic acid release is plotted against the degree of hydrolysis after lipase treatment in soy oil.

### SEQUENCES

SEQ ID NO: 1: Mature amino acid sequence of Lip1 of Candida rugosa.

SEQ ID NO: 2: A codon optimized mature encoding nucleotide sequence of Lip1 of Candida rugosa for expression in Pichia pastoris.

SEQ ID NO: 3: HIS4 gene from Komagataella phaffii strain ATCC 76273.

SEQ ID NO: 4: Nucleotide sequence of the 34 bp FRT recombination site

SEQ ID NO: 5: Glutamine Alanine repeat

SEQ ID NO: 6: α-mating factor from Saccharomyces cerevisiae followed by a Kex2 processing site (KR) and Glutamine Alanine repeat (SEQ ID NO:5)

SEQ ID NO: 7: Nucleotide sequence encoding a Kex2 processing site followed by the Glutamine Alanine repeat and the codon optimized Candida rugosa 534 wild type lipase (LIP1) with an additional XhoI site and NotI site at the 5′ and 3′ ends, respectively.

### DETAILED DESCRIPTION

In one aspect the present invention relates to a polypeptide having lipase activity wherein the polypeptide is selected from the group consisting of
e) a polypeptide, which, when aligned with the polypeptide according to SEQ ID NO: 1, comprises at least the amino acid substitution G414X, wherein the numbering of amino acid position(s) is/are defined with reference to SEQ ID NO: 1, or corresponding position;
f) a polypeptide according to a), wherein the polypeptide has at least 80%, 85%, 90%, 91%, 92%, 93%, 94%, 95%, 96%, 97%, 98%% or 99% identity to the amino sequence of SEQ ID NO: 1;
g) a polypeptide encoded by a nucleic acid which has at least 80%, 85%, 90%, 91%, 92%, 93%, 94%, 95%, 96%, 97%, 98%, or 99% identity to the nucleotide sequence of SEQ ID NO: 2, wherein SEQ ID NO: 2 comprises at least one mutation resulting in at least the amino acid substitution G414X of a polypeptide according to SEQ ID NO: 1 or corresponding position, wherein the numbering of amino acid position(s) is/are defined with reference to SEQ ID NO: 1; and,
h) a polypeptide encoded by a nucleic acid comprising a sequence that hybridizes under low, medium and/or high stringency conditions to the complementary strand of sequence of SEQ ID NO: 2, wherein SEQ ID NO: 2 comprises at least one mutation resulting in at least the amino acid substitution G414X of a polypeptide according to SEQ ID NO: 1 or corresponding position, wherein the numbering of amino acid position(s) is/are defined with reference to SEQ ID NO: 1.

The position in a polypeptide of the invention, which may be a recombinant, synthetic or variant polypeptide, which correspond to the position set out above in SEQ ID NO: 1 may be identified by aligning the sequence of the polypeptide of the present invention with that of SEQ ID NO: 1 using, for example, the alignment by the program Needle, to the most homologous sequence found by the Needle program (see above for details of this program). The positions in the polypeptide of the present invention corresponding to the positions in SEQ ID NO: 1 as set out above may thus be identified and are referred to as those positions defined with reference to SEQ ID NO: 1. Positions of an amino acid substitution are indicated in comparison with SEQ ID NO: 1 wherein Ala (A) at position 1 in SEQ ID NO: 1 is counted as number 1.

A polypeptide as disclosed herein may be an isolated, substantially pure, pure, recombinant, synthetic or variant polypeptide,

Lipase activity as used herein relates to an enzymatic activity that hydrolyses a lipid such as a triacylglycerol, a phospholipid or a galactolipid. For instance, a lipase as disclosed herein may hydrolyse a fatty acid from a triacylglycerol, such as the fatty acids palmitate, eicosapentaenoate (EPA), docosahexaenoate (DHA), oleate and/or linoleate. A lipase as disclosed herein may belong to enzyme classification EC 3.1.1.3.

Lipase specificity relates to a polypeptide having lipase activity where the activity is specified towards a fatty acid side chain of a lipid, for instance lipids with palmitic acid, eicosapentaenoate (EPA), docosahexaenoate (DHA), oleate and/or linoleate. For instance, a lipase specificity towards palmitic acid relates to a lipase having activity towards a lipid wherein at least one of the hydroxyl groups of glycerol is esterified with palmitic acid.

The wording palmitic acid, eicosapentaenoate (EPA), docosahexaenoate (DHA), oleate and/or linoleate, refer to the acid form of these fatty acids and palmitate, eicosapentaenoate and docosahexaenoate refer to the salt and ester form of these fatty acids. The terms may be used interchangeably herein.

Surprisingly, it was found that the ratio of lipase activity on palmitic acid relative to the lipase activity on eicosapentaenoate (EPA), docosahexaenoate (DHA), oleate and/or linoleate of a polypeptide as disclosed herein was higher than this ratio of a corresponding wild type polypeptide. Preferably, the ratio of the lipase activity on palmitic acid relative to the lipase activity on eicosapentaenoic acid (EPA) of a polypeptide as disclosed herein is at least 1.5, 2, 3, 4, 5, 6, 7, 8, 9, 10 or 100 or more times higher than this ratio of a corresponding wild type polypeptide.

Accordingly, in a preferred embodiment, the polypeptide has a higher specificity towards myristate, palmitate and/or stearate relative to the specificity towards eicosapentanoate (EPA) than the specificity towards myristate, palmitate and/or stearate relative to the specificity towards eicosapentanoate of a corresponding wild-type polypeptide and/or wherein the polypeptide has a higher specificity towards palmitate relative to the specificity towards oleate and/or linoleate than the specificity towards palmitate relative to the specificity towards oleate and/or linoleate of a corresponding wild-type polypeptide.

A polypeptide as disclosed herein preferably also has a lower specificity towards DHA than the specificity towards DHA of a corresponding wild type polypeptide.

A corresponding wild type polypeptide is understood to be a polypeptide that does not comprise an amino acid substitution or combination of amino acid substitutions as a polypeptide according to the present disclosure, for instance a polypeptide comprising or consisting of SEQ ID NO: 1.

On one embodiment, the present X in G414X is not G.

In one embodiment a polypeptide as disclosed herein, may be a polypeptide which, when aligned with an amino acid sequence according to SEQ ID NO: 1, comprises at least an amino acid substitution G414T, wherein the numbering of amino acid position(s) is/are defined with reference to SEQ ID NO: 1, wherein preferably the polypeptide has at least 80%, 85%, 90%, 91%, 92%, 93%, 94%, 95%, 96%, 97%, 98%% or 99% identity to the amino sequence of SEQ ID NO: 1.

For instance a polypeptide as disclosed herein may be a variant of the polypeptide or the mature polypeptide of SEQ ID NO:1 comprising at least an amino acid substitution G414T, wherein the numbering of amino acid position(s) is/are defined with reference to SEQ ID NO: 1, wherein the amino acid positions are defined with reference to SEQ ID NO: 1, and further having 1, 2, 3, 4, 5, 6, 7, 8, 9, 10, 11 or 12 further amino substitutions, deletions and/or insertions, whereby the polypeptide still has the activity or function of the polypeptide of the invention. The skilled person will appreciate that these minor amino acid changes in the polypeptide of the invention may be present (for example naturally occurring mutations) or made (for example using r-DNA technology) without loss of the protein function or activity. In case these mutations are present in a binding domain, active site, or other functional domain of the polypeptide a property of the polypeptide may change but the polypeptide may keep its activity. In case a mutation is present which is not close to the active site, binding domain, or other functional domain, less effect may be expected.

In a preferred embodiment, the present X represents a polar uncharged amino acid, preferably chosen from the group consisting of amino acids S, T, N, G, C, Y, and Q. More preferably, the present X is chosen from A, S, T and V. Most preferably, X is T.

In a preferred embodiment, the present polypeptide further comprises one or more amino acid substitutions chosen from the group consisting of I100V, S450A, L413M, L410F, S365A, S365Q, Y361W, F362L, and V409A.

In a preferred embodiment, the present polypeptide comprises amino acid substitutions chosen from the group consisting of:
G414T, G414A, G414S, G414V;
G414T+I100V;
G414T+S450A;
G414T+S450A+I100V;
G414T+L413M;
G414A+L410F;
G414S+L410F;
G414V+L410F;
G414V+F362L;
G414T+V409A; and
G414T+L410F+F362L.

A polypeptide according to the present invention may be derived from any suitable eukaryotic or prokaryotic cell. A eukaryotic cell may be a mammalian, insect, plant, fungal, or algal cell. A prokaryotic cell may be a bacterial cell.

The wording “derived” or “derivable from” with respect to the origin of a polypeptide as disclosed herein, means that when carrying out a BLAST search with a polypeptide according to the present invention, the polypeptide according to the present invention may be derivable from a natural source, such as a microbial cell, of which an endogenous polypeptide shows the highest percentage homology or identity with the polypeptide as disclosed herein

A polypeptide having lipase activity may be derived from any suitable fungi such as from Aspergillus, Rhizomucor, Rhizopus, or Penicillium, for instance Aspergillus niger, A. oryzae, Rhizomucor meihei, Rhizopus microsporus, or Penicillium chrysogenum. A polypeptide having lipase activity may also be derived from yeasts, such as Candida, Kluyveromyces, Pichia, or Saccharomyces, for instance Candida rugosa, Kluyveromyces lactis, Pichia pastoris, or Saccharomyces cerevisiae. A polypeptide having lipase activity may be derived from Candida rugosa.

A polypeptide as disclosed herein may be a naturally occurring polypeptide or a genetically modified or recombinant polypeptide.

A polypeptide as disclosed herein may be purified. Purification of protein is known to a person skilled in the art. A well-known method for purification of proteins is high performance liquid chromatography.

In another aspect the present invention provides a composition comprising a polypeptide as disclosed herein.

A composition as disclosed herein, may comprise a carrier, an excipient, an auxiliary enzyme, or other compounds. Typically a composition, or a formulation, comprises a compound with which a lipase may be formulated, for instance water.

An excipient as used herein is an inactive substance formulated alongside with a polypeptide as disclosed herein, for instance sucrose or lactose, glycerol, sorbitol or sodium chloride. A composition comprising a polypeptide as disclosed herein may be a liquid composition or a solid composition. A liquid composition usually comprises water. When formulated as a liquid composition, the composition usually comprises components that lower the water activity, such as glycerol, sorbitol or sodium chloride (NaCl). A solid composition comprising a polypeptide as disclosed herein may comprise a granulate comprising the enzyme or the composition comprises an encapsulated polypeptide in liquid matrices like liposomes or gels like alginate or carrageenans. There are many techniques known in the art to encapsulate or granulate a polypeptide or enzyme (see for instance G. M. H. Meesters, “Encapsulation of Enzymes and Peptides”, Chapter 9, in N.J. Zuidam and V.A. Nedović (eds.) “Encapsulation Technologies for Active Food Ingredients and food processing” 2010).

A composition as disclosed herein may also comprise a carrier comprising a polypeptide as disclosed herein. A polypeptide as disclosed herein may be bound or immobilized to a carrier by known technologies in the art.

Disclosed herein is also a process for preparing a composition comprising a polypeptide as disclosed herein, which may comprise spray drying a fermentation medium comprising the polypeptide, or granulating, or encapsulating a polypeptide as disclosed herein, and preparing the composition.

Furthermore, the present disclosure relates to a packaging, such as a can, a keg or a barrel comprising a polypeptide or a composition comprising a polypeptide as disclosed herein.

Polypeptides as disclosed herein may be obtained by several procedures known to a skilled person in the art, such as:
1. Error prone PCR to introduce random mutations, followed by a screening of obtained (variant) polypeptides and isolating of (variant) polypeptide(s) with improved kinetic properties
2. Family shuffling of related variants of the genes encoding the polypeptide according to the invention, followed by a screening of obtained variants and isolating of variants with improved kinetic properties

Variants of genes encoding a polypeptide as disclosed herein leading to an increased level of mRNA and/or protein, resulting in more activity may be obtained by modifying the polynucleotide sequences of said genes. Among such modifications are included:
1. Improving the codon usage in such a way that the codons are (optimally) adapted to the parent microbial host.
2. Improving the codon pair usage in such a way that the codons are (optimally) adapted to the parent microbial host
3. Addition of stabilizing sequences to the genomic information encoding a polypeptide according to the invention resulting in mRNA molecules with an increased half life

Methods to isolate variants with improved catalytic properties or increased levels of mRNA or protein are described in WO03/010183 and WO03/01311. Methods to optimize the codon usage in parent microbial strains are for instance described in WO2008/000632. Methods for the addition of stabilizing elements to the genes encoding the polypeptide of the invention are described in WO2005/059149.

Accordingly, in one aspect, a method for generating a variant polypeptide having lipase activity is provided wherein the method comprises
selecting a parent polypeptide comprising at least 80%, 85%, 90%, 91%, 92%, 93%, 94%, 95%, 96%, 97%, 98%, 99% or 100% identity to the amino acid sequence according to SEQ ID NO: 1; and,
substituting at least an amino acid G414T, wherein the numbering of amino acid position(s) is/are defined with reference to SEQ ID NO: 1; and
generating the variant polypeptide, wherein the polypeptide having lipase activity has a higher specificity towards palmitate relative to the specificity towards EPA than specificity towards palmitate relative to the specificity towards EPA of a corresponding wild type polypeptide.

Generating a variant polypeptide as disclosed herein may include expressing a gene encoding the variant polypeptide in a suitable (recombinant) host cell, and cultivating the host cell to generate the variant polypeptide.

In another aspect the present invention relates to a nucleic acid wherein the nucleic acid has at least 80%, 85%, 90%, 95%, 96%, 97%, 98%, or 99% sequence identity to SEQ ID NO: 2, wherein SEQ ID NO: 2 comprises at least one mutation resulting in the amino acid substitution G414X, and optionally one or more amino acid substitutions chosen from the group consisting of I100V, S450A, L413M, L410F, S365A, S365Q, Y361W, F362L, and V409A, of a polypeptide according to SEQ ID NO: 1, wherein the numbering of amino acid position(s) is/are defined with reference to SEQ ID NO: 1.

A nucleic acid sequence as disclosed herein may be a codon optimized, or a codon pair optimized sequence for optimal expression of a polypeptide as disclosed herein in a particular host cell.

In one embodiment a nucleic acid is disclosed that is an isolated, substantially pure, pure, recombinant, synthetic or variant nucleic acid of the nucleic acid as disclosed herein.

In another embodiment, a nucleic acid molecule of the invention comprises a nucleic acid molecule which is the reverse complement of the nucleotide sequence shown in SEQ ID NO: 2, wherein SEQ ID NO: 2 comprises at least one mutation resulting in an amino acid substitution G414X of a polypeptide according to SEQ ID NO: 1, wherein the numbering of amino acid position(s) is/are defined with reference to SEQ ID NO: 1.

Preferably, X is chosen from the group consisting of A, S, T and V.

Also disclosed is a nucleic acid that hybridizes under medium stringency, preferably under high stringency conditions to the complementary strand of the mature polypeptide coding sequence of SEQ ID NO:2, wherein SEQ ID NO: 2 comprises at least one mutation resulting in an amino acid substitution G414T of a polypeptide according to SEQ ID NO: 1, wherein the numbering of amino acid position(s) is/are defined with reference to SEQ ID NO: 1

In one aspect the present disclosure relates to an expression vector comprising a nucleic acid as disclosed herein operably linked to at least one control sequence that directs expression of the polypeptide in a host cell.

There are several ways of inserting a nucleic acid into a nucleic acid construct or an expression vector which are known to a person skilled in the art, see for instance Sambrook & Russell, Molecular Cloning: A Laboratory Manual, 3rd Ed., CSHL Press, Cold Spring Harbor, N.Y., 2001. It may be desirable to manipulate a nucleic acid encoding a polypeptide of the present invention with control sequences, such as promoter and terminator sequences.

A promoter may be any appropriate promoter sequence suitable for a eukaryotic or prokaryotic host cell, which shows transcriptional activity, including mutant, truncated, and hybrid promoters, and may be obtained from polynucleotides encoding extracellular or intracellular polypeptides either endogenous (native) or heterologous (foreign) to the cell. The promoter may be a constitutive or inducible promoter. Preferably, the promoter is an inducible promoter, for instance a starch inducible promoter. Promoters suitable in filamentous fungi are promoters which may be selected from the group, which includes but is not limited to promoters obtained from the polynucleotides encoding A. oryzae TAKA amylase, Rhizomucor miehei aspartic proteinase, Aspergillus gpdA promoter, A. niger neutral alpha-amylase, A. niger acid stable alpha-amylase, A. niger or A. awamori glucoamylase (glaA), A. niger or A. awamori endoxylanase (xInA) or beta-xylosidase (xInD), T. reesei cellobiohydrolase I (CBHI), R. miehei lipase, A. oryzae alkaline protease, A. oryzae triose phosphate isomerase, A. nidulans acetamidase, Fusarium venenatum amyloglucosidase (WO 00/56900), Fusarium venenatum Dania (WO 00/56900), Fusarium venenatum Quinn (WO 00/56900), Fusarium oxysporum trypsin-like protease (WO 96/00787), Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the polynucleotides encoding A. niger neutral alpha-amylase and A. oryzae triose phosphate isomerase), and mutant, truncated, and hybrid promoters thereof.

Any terminator which is functional in a cell as disclosed herein may be used, which are known to a person skilled in the art. Examples of suitable terminator sequences in filamentous fungi include terminator sequences of a filamentous fungal gene, such as from Aspergillus genes, for instance from the gene A. oryzae TAKA amylase, the genes encoding A. niger glucoamylase (glaA), A. nidulans anthranilate synthase, A. niger alpha-glucosidase, trpC and/or Fusarium oxysporum trypsin-like protease.

In another aspect the present disclosure relates to a host cell comprising a nucleic acid or an expression vector as disclosed herein. A suitable host cell may be a mammalian, insect, plant, fungal, or algal cell, or a bacterial cell. A suitable host cell may be a fungal cell, for instance from the genus Acremonium, Aspergillus, Chrysosporium, Fusarium, Myceliophthora, Penicillium, Rasamsonia, Talaromyces, Thielavia, Trichoderma, Saccaromyces, Kluyveromyces, Pichia, for instance Aspergillus niger, Aspergillus awamori, Aspergillus foetidus, A. oryzae, A. sojae, Talaromyces emersonii, Rasamsonia emersonii Chrysosporium lucknowense, Fusarium oxysporum, Myceliophthora thermophila, Thielavia terrestris or Trichoderma reesei or, Saccharomyces cerevisiae, Kluyveromyces lactis, Pichia pastoris. A host cell may be Pichia pastoris.

A host cell may be a recombinant or transgenic host cell. The host cell may be genetically modified with a nucleic acid or expression vector as disclosed herein with standard techniques known in the art, such as electroporation, protoplast transformation or conjugation for instance as disclosed in Sambrook & Russell, Molecular Cloning: A Laboratory Manual, 3rd Ed., CSHL Press, Cold Spring Harbor, N.Y., 2001. A recombinant host may overexpress a polypeptide according to the present disclosure by known techniques in the art.

In one aspect the present disclosure relates to a process for the production of a polypeptide as disclosed herein comprising cultivating a host cell in a suitable fermentation medium under conditions conducive to the production of the polypeptide and producing the polypeptide. A skilled person in the art understands how to perform a process for the production of a polypeptide as disclosed herein depending on a host cell used, such as pH, temperature and composition of a fermentation medium. Host cells can be cultivated in microtritre plates (MTP), shake flasks, or in fermenters having a volume of 0.5 or 1 litre or larger to 10 to 100 or more cubic metres. Cultivation may be performed aerobically or anaerobically depending on the requirements of a host cell.

Advantageously a polypeptide as disclosed herein is recovered or isolated from the fermentation medium. Recovering or isolating a polypeptide from a fermentation medium may for instance be performed by centrifugation, filtration, and/or ultrafiltration, or chromatography.

In one aspect the present disclosure relates to a process for preparing a product comprising an oil or fat comprising bringing an intermediary form of the product comprising oil or fat into contact with a polypeptide or a composition as disclosed herein and preparing the product.

A product that may be prepared in a process as disclosed herein may be a food or feed product, for instance a food or feed product comprising fish oil or soy oil. A food or feed product disclosed herein may be fish oil or soy oil. Fish oil as disclosed herein may be oil derived from any suitable fish for instance from salmon, mackerel, herring and/or sardine. Oil or fat in a product and/or an intermediary form of a product disclosed herein, for instance fish oil, comprise(s) lipids, such as triacylglycerol comprising at least one palmitate as a side chain. Oil or fat in a product as disclosed herein may further comprise a triacylglycerol comprising eicosapentaenoic acid (EPA) and/or docosahexaenoic acid (DHA) as a side chain. Accordingly, an oil or fat may comprise palmitate, eicosapentaenoate, docosahexaenoate (DHA) oleate and/or linoleate.

Bringing an intermediary form of the product comprising oil or fat into contact with a polypeptide as disclosed herein may comprise mixing or stirring a polypeptide having lipase activity with the oil or fat. An intermediary form of a product in a process as disclosed herein may comprise water.

Said bringing in contact may also comprise adding water to the intermediary form of the product. Bringing oil or fat into contact with a polypeptide having lipase activity may further comprise incubating the polypeptide with the oil or fat at a suitable temperature and pH. A suitable temperature may for instance be between 10 and 70 degrees Celsius, such as between 15 and 65 degrees Celsius, for instance between 20 and 60 degrees Celsius, for instance between 25 and 50 degrees Celsius. A suitable pH may be a pH between 3.5 and 9, for instance between 4 and 8, for instance between 4.5 and 7.5. Bringing oil and or fat into contact with a polypeptide having lipase activity may include hydrolysing a triacylglycerol comprising at least one palmitate as a side chain.

A process for preparing a product comprising oil or fat may further comprise separating a fatty acid from the product comprising oil or fat. Fatty acids may be an aqueous phase comprising a fatty acid. A fatty acid may be palmitic acid. Separating a fatty acid, for instance an aqueous phase comprising a fatty acid, may comprise centrifugation or filtration. Also disclosed herein is a product comprising oil or fat obtainable by a process as disclosed here in.

In one aspect the present disclosure relates to the use of a polypeptide as disclosed herein to lower saturated fatty acids and/or monounsaturated fatty acids in an oil or fat. Preferably, the oil is chosen from fatty acid ester oil, triglyceride oil and fatty acid ethyl ester oil. Preferably, wherein the fatty acids are chosen from the group consisting of lauric acid (C12:0), myristic acid (C14:0) myristoleic acid (C14:1), palmitic acid (C16:0), palmitoleic acid (C16:1), stearic acid (C18:0), oleic acid (C18:1), arachidic acid (C20:0), 11-eicosenoic acid or gondoic acid (C20:1), docosanoic acid (C22:0) and erucic acid or brassidic acid (C22:1). Lowering monounsaturated or saturated fatty acids in an oil or fat means that an amount of saturated fatty acids in an oil or fat is reduced. The amount of saturated fatty acids that is reduced by a polypeptide having lipase activity as disclosed herein is lower than the amount of saturated fatty acids that is reduced by a corresponding wild type polypeptide. An oil as used herein may be a fish oil or soy oil. More preferably, the oil is chosen from the group consisting fish oil, soy oil, sunflower oil, safflower oil, grapeseed oil, flaxseed oil and walnut oil.

Accordingly, disclosed herein is a process for reducing an amount of saturated fatty acids or monounsaturated fatty acids in an oil or fat, comprising incubating the oil or fat with a polypeptide having lipase activity as disclosed herein. Incubating on oil or fat with a polypeptide having lipase activity as disclosed herein may be performed as disclosed herein above.

The following examples illustrate the invention.

### EXAMPLES

### Materials and Methods

### Strains

Pichia pastoris (Komagataella phaffii) (strain ATCC 76273/CBS 7435/CECT 11047/NRRL Y-11430/Wegner 21-1) was used (Cregg J M, Barringer K J, Hessler A Y and Madden K R (1985). Pichia pastoris as a host system for transformations. Mol.Cell. Biol., 5, 3376-3385).

### Molecular Biology Techniques

Molecular biology techniques were performed according to (Sambrook & Russell, Molecular Cloning: A Laboratory Manual, 3rd Ed., CSHL Press, Cold Spring Harbor, N.Y., 2001). PCR is disclosed in for example Innes et al. (1990) PCR protocols, a guide to methods and applications, Academic Press, San Diego. Polymerase chain reaction (PCR) was performed on a thermocycler with Phusion High-Fidelity DNA polymerase (Finnzymes O Y, Aspoo, Finland) according to the instructions of the manufacturer.

Standard DNA procedures were carried out as described in Sambrook & Russell, 2001, Molecular cloning: a laboratory manual, 3rd Ed., Cold Spring Harbor Laboratory Press, Cold Spring Harbor, N.Y. unless otherwise stated. DNA sequences were ordered at DNA 2.0 (CA, USA).

### Determination of Free Fatty Acids

The amounts of Free Fatty Acids (FFA) were determined with a gas chromatograph (GC) using a split injector and a Flame Ionization Detector (FID). The method was based on the analysis of FFA in Milk and Cheese described by C. de Jong, H. T. Badings, (1990), Journal of High Resolution Chromatography, Vol 13, p. 94-98, and the official AOCS method Ce 1h-05, (2009) Determination of cis-, trans-, Saturated, Monounsaturated and Polyunsaturated Fatty Acids in Vegetable or Non-Ruminant Animal oils and Fats by Capillary GLC, with adaptations as described below.

An Agilent 7890 GC was equipped with a Supelco SP™ 2560 (Sigma-Aldrich) capillary column (100 m×0.25 mm, df=0.2 μm). Hydrogen was used as the carrier gas at a constant flow rate of 1.3 mL/min with a split flow of 32.5 ml/min. During the analysis, the oven temperature was initially set at 170° C. and after 30 minutes raised to 240° C. at a rate of 5° C./min. The injector temperature was set to 250° C. and the detector temperature was set to 325° C. Pentadecanoic acid dissolved in chloroform (2 mg/ml) was used as internal standard. Calibration lines were made with external standards of the FFA. The complete sample (4 g oil and buffer) was mixed with 10 ml of the internal standard solution. After centrifugation, 0.25 ml of the chloroform layer was applied to an amino propyl solid phase extaction (SPE) column (Bond Elut, 500 mg), conditioned with 10 ml n-heptane. The SPE was rinsed with 5 ml chloroform/2-propanol (1:1). The FFA were eluted with 5 ml diethyl ether containing 2% formic acid. The FFA fraction was subsequently methyl esterfied with a boron trifluoride—methanol solution according to W.R. Morrison and L. M. Smith, (1964), Journal of Lipid Research, Vol. 5, pp. 600-608. After extraction with n-heptane, 1 μL of the n-heptane layer was injected into the GC.

The peak areas of the FFA were normalized with the peak area of the internal standard. The amounts of FFA were calculated by interpolation of the normalized peak areas of the FFA with the calibration curves of the normalized external standards. The amount of FFA was expressed as μg/g.

### Example 1

1.1. Preparation of Histidine Auxotrophic Pichia pastoris (Komagataella phaffii) Strain

The HIS4 gene (SEQ ID NO: 3) from Pichia pastoris strain ATCC 76273 was deleted by using a FLP recombinase and two asymmetric FLP recombination target sequences (FRTs) derived from S. cerevisiae 2 μm circle (Som, T., Armstrong, K. A., Volkert, F. C., and Broach, J. R. (1988), Cell 52: p. 27-37; Broach, J. R. (1981) The yeast plasmid 2 μm circle. In: The molecular biology of the yeast Saccharomyces: Life cycle and inheritance. Strathern, J. N., Jones, E. W., and Broach, J. R. (eds)., Cold Spring Harbor, pp. 455-470). This resulted in a histidine auxotrophic strain DSM101A wherein the 2682 bp HIS4 open reading frame (SEQ ID NO: 3) was replaced with a 34 bp FRT recombination site (SEQ ID NO: 4). The HIS4 deletion was confirmed by Southern analyses and phenotypically. The histidine auxotrophic strain DSM101A was not able to grow on MD media (Sambrook & Russell) without histidine, whereas this strain grew well on MD media with histidine (40 μg/ml).

MD contains 15 g/L agar, 800 mL H₂O, and after autoclaving the following filter sterilized solutions were added: 100 mL 10×YNB (134 g/L Difco™ Yeast Nitrogen Base w/o Amino Acids), 2 mL 500×B (0.02% D-Biotin), 100 mL 10×D (220 g/L α-D(+)-Glucose monohydrate).

1.2. Preparation of variant lipase DNA construct

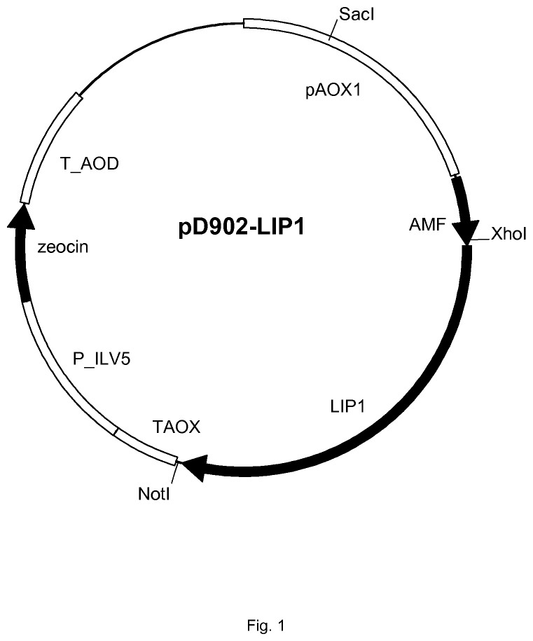

The Pichia expression vector pD902 (DNA2.0, CA, USA) was used for expression of mature Candida rugosa 534 lipase polypeptide variants (variants of amino acids 1-534 of SEQ ID NO: 1). The lipase encoding sequences were fused behind the α-mating factor from S. cerevisiae followed by a Kex2 processing site composed of Lysine, Arginine (KR) and a Glutamine Alanine repeat (EAEA) (SEQ ID NO: 5) The genes were placed under control of the methanol inducible AOX1 promoter as described previously (Brocca S., Schmidt-Dannert C., Lotti M., Alberghina L., Schmid R.D., Protein Sci. 1998(6):1415-1422) and WO9914338A1. The Candida rugosa 534 wild type lipase polypeptide sequence (SEQ ID NO: 1) was used to design a nucleotide sequence encoding the lipase with a codon usage that matched the coding usage of Pichia pastoris (SEQ ID NO: 2). Additionally, a XhoI site was placed at the 5′ end and a NotI site at the 3′ end. The nucleotide sequence comprising the codon optimized gene fragment encoding the Candida rugosa 534 wild type sequence (LIP1), the α-mating factor from S. cerevisiae followed by a Kex2 processing site composed of Lysine, Arginine (KR) and a Glutamine Alanine repeat (EAEA) and a XhoI site at the 5′ end and a NotI site at the 3′ end is shown in SEQ ID NO: 7. The pD902 vector with SEQ ID NO: 7 is depicted in FIG. 1.

Variant of the LIP1 protein (SEQ ID NO: 1) was made with the amino acid substitution G414T. Position of the amino acid substitution is indicated in comparison with SEQ ID NO: 1 wherein Ala (A) at position 1 in SEQ ID NO: 1 is counted as number 1.

The LIP1 encoding gene variant containing the amino acid substitution G414T was cloned into vector pD902 following the procedure as described above for the LIP1 encoding wild type sequence. The pD902 vectors containing the lip1 gene variants were digested by SacI and transformed to Pichia pastoris strain DSM101A. Transformation procedure was performed according to condensed electroporation protocol using freshly prepared solutions (Lin-Cereghino J1, Wong W W, Xiong S, Giang W, Luong L T, Vu J, Johnson S D, Lin-Cereghino G P. Biotechniques. (2005) 38, (1):44-48). Transformants were plated on YPDS agar plates with 500 μg/mL Zeocin (YPDS: 1% yeast extract, 2% peptone, 2% glucose, 1 M sorbitol, 2% agar) and incubated at 30° C. for 72h.

### Example 2

### Production of Lipase Variants

Histidine auxotrophic Pichia pastoris clones containing a LIP1 variant with amino acid substitution G414T and G414T in combination with one or more of I100V, S450A, L413M, L410F, S365A, S365Q, Y361W, F362L, and V409A were cultured in 1.5 mL BMD 1% medium (0.2M Potassium Phosphate buffer, 13.4 g/I Yeast Nitrogen Base, 0.4 mg/mL biotin, 11 g/L glucose, filter sterilized) in 24 deep wells plates (Axygen, California, USA). These cultures were incubated for 60 hours at 28° C., 550 rpm (Microton incubator shaker (Infors AG, Bottmingen, Switzerland). After 60 hours of incubation, 1.25 mL BMM2 (0.2 M Potassium Phosphate buffer, 13.4 g/L Yeast Nitrogen Base, 0.4 mg/ml Biotin, 1% methanol, filter sterilized) was added and growth was continued at 28° C., 550 rpm. After 8 hours, 250 μL BMM10 (0.2 M Potassium Phosphate buffer, 13.4 g/L Yeast Nitrogen Base, 0.4 mg/ml Biotin, 5% methanol, filter sterilized) was added to induce lipase production. Addition of 250 μL BMM10 was repeated after 24 hours, 48 hours and 72 hours after the first addition. 12 hours after the last addition of BMM10, the cultures were centrifuged (5 min, 1000 g) and supernatants were harvested and stored at −20° C.

### Example 3

### Screening of Lipase Activity on p-NP Substrates

The activity of the LIP1 variant comprising mutation G414T was determined in assays using the chromogenic substrates: 4-nitrophenyl Palmitate (Sigma N2752), 4-nitrophenyl Oleate (custom made by Syncom), 4-nitrophenyl Linoleate (custom made by Syncom), 4-nitrophenyl eicosa 5,8,11,14,17-penta enoate (EPA, custom made by Syncom) and 4-nitrophenyl docosa 4,7,10,13,16,19-hexa enoate (DHA, custom made by Syncom). An 8.0 mM solution of the chromogenic substrates in 2-propanol was made. Subsequently, 3.5 mL of this solution was added to 46.5 mL 100 millimol/L sodium acetate buffer pH 4.5 containing 1% Triton X-100, under vigorously stirring. The enzyme reaction was started by mixing 25 μL of a suitable dilution of the broth supernatant prepared as described above with 225 μL substrate solution (substrate concentration during incubation is 0.5 mM) in a microtiter plate using the Hamilton robot. 200 μL of the reaction mixture was transferred by the Hamilton robot into an empty microtiter plate which was put into a TECAN Infinite M1000 micro titer plate reader. During the incubation at 25° C., the change in absorption of the mixture was measured for 20-60 minutes at 348 nm (isosbestic point of 4-nitrophenol). The slope (deltaOD/min) of the linear part of the curve is used as measure for the activity.

The activity is expressed as the amount of enzyme that liberates 1 micro p-nitrophenol per minute under the conditions of the test. Samples were diluted such to assure that the absorbance increase after the incubation is less than 0.7. Calibration is done using a 4-nitrophenol standard solution (Sigma N7660) diluted in the same buffer.

Table 1 and 2 show the activity of LIP1 variant with mutation G414T and the wild type Lip1 lipase on the substrates pNP-Palmitate, pNP-DHA and pNP-EPA, wherein the strains are grown in MTP and shake flask, respectively. The ratio of palmitate hydrolysing activity vs EPA hydrolysing activity of all mutant Lip1 variant was increased compared to the wild type enzyme both when grown in MTP and shake flasks.

Table 3 show the activity of LIP1 variants and the wild type Lip1 lipase on the substrates pNP-palmitate, pNP-oleate and pNP-linoleate, wherein the strains are grown in shake flask. The ratio of palmitate hydrolysing activity vs oleate and linoleate hydrolysing activity of the mutant Lip1 variants was increased compared to the wild type enzyme both, indicating an improved specificity for hydrolysing palmitic acid.

TABLE 1
Activity of LIP 1 variants and LIP1 wild type expressed in Pichia
pastoris after growth of the strains in MTP, on pNP-Palmitate
(Palmitase), pNP-DHA (DHAase) and pNP-EPA (EPAase) as
substrate measured at pH 4.5 and 25° C. and ratio of activities.

| | | Palmi- | DHAase | EPAase | | |
|---|---|---|---|---|---|---|
| | | tase (P) | (D) | (E) | | |
| | Muta- | (μmol/ | (μmol/ | (μmol/ | Ratio | Ratio |
| Strain | tion | min · ml) | min · ml) | min · ml) | P/D | P/E |
| CR_LIP1 | LIP1 | 0.95 | 0.0007 | 0.097 | 1356 | 10 |
| | wild | | | | | |
| | type | | | | | |
| CRL_060 | G414T | 0.28 | 0.0003 | 0.002 | 917 | 138 |

TABLE 2
Activity of LIP 1 variant and the LIP1 wild type expressed in
Pichia pastoris after growth of the strains in shake flasks, on
pNP-Palmitate (Palmitase), pNP-DHA (DHAase) and
pNP-EPA (EPAase) as substrate measured at pH 4.5
and 25° C. and ratio of activities.

| | | Palmi- | DHAase | EPAase | | |
|---|---|---|---|---|---|---|
| | | tase (P) | (D) | (E) | | |
| | Muta- | (μmol/ | (μmol/ | (μmol/ | Ratio | Ratio |
| Strain | tion | min · ml) | min · ml) | min · ml) | P/D | P/E |
| CR_LIP1 | LIP1 | 8.43 | 0.0330 | 4.26 | 255 | 2 |
| | wild | | | | | |
| | type | | | | | |
| CRL_060 | G414T | 2.83 | 0.0090 | 0.16 | 314 | 18 |

TABLE 3
Ratio of activity of LIP1 variants and LIP1 wild type expressed in Pichia pastoris after
growth of the strains in shake flask, on pNP-Palmitate (Palmitase), pNP-Oleate (Olease) and
pNP-Linoleate (Linolease) as substrate measured at pH 4.5 and 25° C. P/O = ratio of activities on
pNP-palmitate and pNP-oleate. P/L = ratio of activities on pNP-palmitate and pNP-linoleate.
Increased P/O- and P/L ratios compared to the wild type LIP1 lipase indicates an improved
specificity towards the hydrolysis of palmitic acid.

| | | | | | | | | | | Activity | |
|---|---|---|---|---|---|---|---|---|---|---|---|
| | Mutation (position number) | | | | | | | | | ratio | |
| Variant | 100 | 361 | 362 | 365 | 409 | 410 | 413 | 414 | 450 | P/O | P/L |
| CRL_060 | | | | | | | | G414T | | 2.1 | 1.8 |
| CR2L_013 | I100V | | | | | | | G414T | | 1.9 | 2.1 |
| CR2L_014 | | | | | | | | G414T | S450A | 0.9 | 1.4 |
| CR2L_015 | I100V | | | | | | | G414T | S450A | 1.5 | 1.4 |
| CR2L_019 | | | | | | | L413M | G414T | | 2.1 | 2.2 |
| CR3L_007 | | | | | | L410F | | G414A | | 4.5 | 2.8 |
| CR3L_008 | | | | | | L410F | | G414S | | >10 | 6.2 |
| CR3L_009 | | | | | | L410F | | G414V | | 3.8 | 2.3 |
| CRL3_20 | | Y361W | | S365A | | | | G414T | | >10 | >10 |
| CRL3_24 | | Y361W | | S365A | | | | G414P | | >10 | 3.8 |
| CR3L_053 | | | F362L | | | | | G414V | | 3.8 | 6.7 |
| CR3L_054 | | | | | V409A | | | G414T | | 2.7 | 3.3 |
| CR3L_058 | | | F362L | | | L410F | | G414T | | >10 | 5.9 |
| CR3L_059 | | | | | V409A | L410F | | G414T | | 2.9 | 2.1 |
| WT LIP 1 | | | | | | | | | | 0.9 | 0.9 |

### Example 4

### Lipase Activity in Fish Oil

The activity of the LIP1 variant with mutation G414T, G414T with I100V and/or with S450A was compared with the activity of wild type LIP1 lipase in an application type incubation on fish oil (Semi-refined fish oil, Ocean nutrition, see table 4 for the composition). 2 mL of enzyme solution diluted in 100 mM phosphate buffer pH 7 of LIP1 mutant G414T and wild type LIP1 was added to 2 ml off fish oil in. After 16 hours of incubation in a water bath at 37° C. under stirring (500 rpm), the reaction was stopped by storing the reaction mixtures at minus 18 degrees Celsius. The fatty acids released were analyzed after extraction of the samples with chloroform using FAME according to the method disclosed above.

TABLE 4
fatty acid composition of fish oil

| | FA | FFA | | MW |
|---|---|---|---|---|
| type of fatty acid | (mg/g oil) | (μmol/g) | mol % | (g/mol) |
| C14:0 | 82 | 359.1 | 12.6 | 228.4 |
| C16:0 | 148 | 577.2 | 20.2 | 256.4 |
| C16:1 | 92 | 361.6 | 12.6 | 254.4 |
| C17:0 | 4 | 14.8 | 0.5 | 270.5 |
| C18:0 | 27 | 94.9 | 3.3 | 284.5 |
| C18:1 | 105 | 371.7 | 13.0 | 282.5 |
| C18:2 | 36 | 128.3 | 4.5 | 280.5 |
| C18:3 | 7 | 25.1 | 0.9 | 278.5 |
| C20:0 | 2 | 6.4 | 0.2 | 312.5 |
| C20:1 | 7 | 22.5 | 0.8 | 310.5 |
| C20:3 | 2 | 6.5 | 0.2 | 306.5 |
| C20:4 | 9 | 31.9 | 1.1 | 282.5 |
| C20:5 (EPA) | 174 | 575.1 | 20.1 | 302.5 |
| C 22:2 | 7 | 20.8 | 0.7 | 336.6 |
| C 22:4 | 0 | 0.0 | 0.0 | |
| C 22:6 (DHA) | 83 | 251.1 | 8.8 | 330.6 |
| C24:1 | 4 | 12.2 | 0.4 | 328.6 |
| sum | 789 | 2859 | 100 | |

The data in table 5 below show that incubation of fish oil with the LIP1 variants resulted in an increased release (mol %) of saturated fatty acids like myristic acid (C14:0), palmitic acid (C16:0), stearic acid (C18:0) and a decreased release (mol %) of EPA (C20:5) compared to the wild type LIP1. The release (mol %) of DHA (C22:6) after incubation of fish oil with LIP1 variants and wild type LIP1 was similar.

TABLE 5
Fatty acids released (mol %) and total amount (μmol/g) after incubation of wild type
LIP1 lipase and LIP1 lipase variants on fish oil.
The maximum total amount of fatty acids that could be liberated from
the fish oil in this experiment was 2859 μmol/gram fish oil.

| | | | | | | | G414T + |
|---|---|---|---|---|---|---|---|
| | | | | | G414T + | G414T + | S450A + |
| | | | WT | G414T | I100V | S450A | I100V |
| Fatty | C14:0 | mol % | 13.7 | 18.6 | 20.1 | 21.1 | 19.7 |
| acid | C16:0 | mol % | 23.5 | 31.8 | 37.9 | 41.4 | 34.1 |
| composition | C16:1 | mol % | 12.3 | 14.1 | 18.0 | 17.1 | 18.3 |
| | C17:0 | mol % | 0.6 | 0.8 | <dl | <dl | <dl |
| | C18:0 | mol % | 3.8 | 5.1 | 5.2 | 6.8 | 5.4 |
| | C18:1 | mol % | 12.6 | 13.5 | 11.0 | 8.5 | 13.0 |
| | C18:2 | mol % | 1.5 | 1.2 | 0.7 | 0.0 | 1.0 |
| | C18:3 | mol % | 0.9 | 0.8 | 0.7 | 0.0 | 1.0 |
| | C20:1 | mol % | 0.3 | 0.4 | <dl | <dl | <dl |
| | C20:5 | mol % | 17.5 | 3.3 | <dl | <dl | <dl |
| | (EPA) | | | | | | |
| | C 22:2 | mol % | 0.2 | 0.0 | 0.0 | <dl | <dl |
| | C 22:4 | mol % | 0.2 | 0.0 | 0.6 | 1.6 | 0.5 |
| | C 22:6 | mol % | 0.2 | 0.0 | <dl | <dl | <dl |
| | (DHA) | | | | | | |
| | unknown | mol % | 12.4 | 10.4 | 5.9 | 3.4 | 7.0 |
| | Total | μmol/g | 1211 | 894 | 545 | 207 | 710 |
| | amount | | | | | | |
| | fatty acids | | | | | | |
| | formed | | | | | | |

Table 6 below shows the effect of enzyme treatment on the composition (calculated from the mass balance) of the refined oil in comparison with non-treated fish oil. It is clearly shown that treatment with the variants results in enriched DHA and EPA content in combination with clear reduced release of EPA when compared the wild type LIP1.

TABLE 6
The effect of enzyme treatment on the composition of the refined oil is shown in this table.
The use of the selected variants results in enriched DHA and EPA content in combination
with clearly reduced release of EPA when compared the wild type LIP1. Composition of
enzyme treated fish oil calculated from mass balance. DH = degree of hydrolysis

| incubation | DH | EPA + DHA | EPA | DHA | EPA | DHA | EPA | DHA |
|---|---|---|---|---|---|---|---|---|
| with | (%) | (mol %) | (mol %) | (mol %) | loss % | loss % | (w/w %) | (w/w %) |
| G414T | 31.3 | 40.5 | 27.8 | 12.8 | 5.2 | 0.0 | 21.5 | 10.8 |
| G414T + | 19.1 | 35.6 | 24.7 | 10.9 | 0.6 | 0.0 | 20.1 | 9.7 |
| I100V | | | | | | | | |
| G414T + | 7.2 | 31.0 | 21.6 | 9.5 | 0.6 | 0.0 | 18.3 | 8.8 |
| S450A | | | | | | | | |
| G414T + | 24.8 | 38.2 | 26.6 | 11.7 | 0.6 | 0.0 | 21.2 | 10.2 |
| I100V + | | | | | | | | |
| S450A | | | | | | | | |
| Wild type | 42.4 | 37.1 | 22.1 | 15.1 | 36.8 | 1.2 | 16.3 | 12.2 |
| buffer | 0 | 28.9 | 20.1 | 8.8 | 0.0 | 0.0 | 17.4 | 8.3 |

### Example 5

### Lipase Activity in Soy Oil

The activity of the LIP1 variants with mutation as indicated in table 8 were compared with the activity of wild type LIP1 lipase in incubation on soy oil (Salad oil from Goldsun, see table 5 for the composition). 2 mL of enzyme solution diluted in 100 mM phosphate buffer pH 7 of LIP1 mutant and wild type LIP1 was added to 2 ml of soy oil. After 16 hours of incubation in a water bath at 37° C. under stirring (500 rpm), the reaction was stopped by storing the reaction mixtures at minus 18 degrees Celsius. The fatty acids released were analyzed after extraction of the samples with chloroform using FAME according to the method disclosed above.

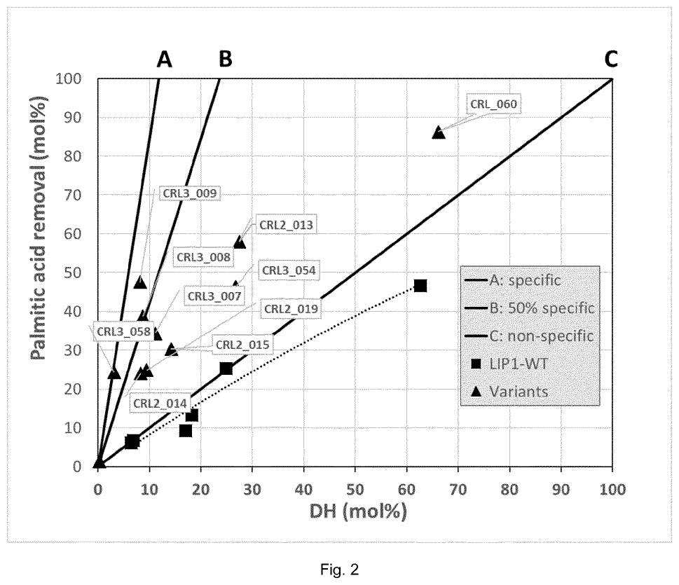

Results are shown in table 6 and FIG. 2. Table 6 shows the degree of hydrolysis (DH) and amounts of palmitic acid released of several experiments with samples produced in shake flask. In FIG. 1, the degree of hydrolysis (DH) is plotted against the amounts of palmitic acid released. When an improved variant is 100% specific for palmitic acid, then the DH versus palmitic acid release should follow line A. In case of a non-specific lipase this is line C. In case of 50% specificity this is line B. The results of the WT LIP1 (circles) all are below line C, indicating a preference for hydrolysing unsaturated fatty acid from soy oil. For variant L410F/S365Q all points are between the B and C line indicating an improved specificity towards palmitic acid when compared with the wild type LIP1.

TABLE 7
Fatty acid composition of soy oil

| | FA | FA | FA | MW |
|---|---|---|---|---|
| fatty acid | (mg/g oil) | (mol %) | (μmol/g) | (g/mol) |
| C14:0 | 1.0 | 0.1 | 4.4 | 228.4 |
| C16:0 | 106 | 11.8 | 413 | 256.4 |
| C16:1 | 1.0 | 0.1 | 3.9 | 254.4 |
| C18:0 | 41.0 | 4.1 | 144 | 284.5 |
| C18:1 | 233 | 23.5 | 825 | 282.5 |
| C18:2 | 518 | 52.5 | 1847 | 280.5 |
| C18:3 | 69.0 | 7.0 | 248 | 278.5 |
| C20:0 | 3.0 | 0.3 | 9.6 | 312.5 |
| C20:1 | 2.0 | 0.2 | 6.4 | 310.5 |
| C22:0 | 3.0 | 0.25 | 8.8 | 340.6 |
| C24:0 | 4.0 | 0.31 | 10.9 | 368.6 |
| total | 982 | 100.0 | 3516 | |

Results of experiments of different LIP1 variants produced in shake flask after incubation with soy oil for 16 h at 37° C. Degree of hydrolysis (DH) and amounts of palmitic acid released are given in the table 8 below. Different types of substrate were use: 50% oil mixed with buffer or 1% oil mixed with 100 mM phosphate buffer pH 7 or 1% soy oil emulsified with 1% triton X-100 mixed in the same buffer. DH=degree of hydrolysis in mol %. P/DH=ratio of the amount of released palmitic acid and degree of hydrolysis. Improved specificity of a variant for palmitic acid hydrolysis in soy oil triglycerides is shown when the P/DH ratio is higher compared the ratio found for the wild type LIP1 lipase.

TABLE 8

| | | | | | | | | | palmitic acid | | |
|---|---|---|---|---|---|---|---|---|---|---|---|
| | Pos | Pos | Pos | Pos | Pos | Pos | Pos | | release | | |
| variant | 100 | 362 | 409 | 410 | 413 | 414 | 450 | substrate | (mol %) | DH (%) | P/DH |
| CRL_060 | | | | | | G414T | | 50% soy oil | 87.3 | 65.3 | 1.3 |
| | | | | | | | | 50% soy oil | 86.3 | 66.2 | 1.3 |
| | | | | | | | | 50% soy oil | 86.2 | 59.9 | 1.4 |
| CRL2_013 | I100V | | | | | G414T | | 50% soy oil | 28.5 | 11.2 | 2.6 |
| | | | | | | | | 50% soy oil | 58.0 | 27.5 | 2.1 |
| CRL2_014 | | | | | | G414T | S450A | 50% soy oil | 24.1 | 8.3 | 2.9 |
| CRL2_015 | I100V | | | | | G414T | S450A | 50% soy oil | 30.4 | 14.2 | 2.1 |
| CRL2_019 | | | | | L413M | G414T | | 50% soy oil | 24.9 | 9.4 | 2.6 |
| CRL3_007 | | | | L410F | | G414A | | 1% soy oil | 34.2 | 11.2 | 3.1 |
| CRL3_008 | | | | L410F | | G414S | | 1% SO/1% triton | 38.7 | 8.7 | 4.4 |
| CRL3_009 | | | | L410F | | G414V | | 1% soy oil | 9.8 | 1.6 | 6.1 |
| | | | | | | | | 1% SO/1% triton | 47.6 | 8.2 | 5.8 |
| CRL3_053 | | F362L | | | | G414V | | 50% soy oil | 22.1 | 4.0 | 5.5 |
| | | | | | | | | 1% SO/1% triton | 47.5 | 23.3 | 2.0 |
| CRL3_054 | | | V409A | | | G414T | | 50% soy oil | 1.1 | 0.2 | 4.9 |
| | | | | | | | | 1% soy oil | 8.2 | 5.6 | 1.5 |
| | | | | | | | | 1% SO/1% triton | 46.3 | 26.8 | 1.7 |
| CRL3_058 | | F362L | | L410F | | G414T | | 1% SO/1% triton | 24.4 | 3.1 | 7.8 |
| CRL3_059 | | | V409A | L410F | | G414T | — | 1% soy oil | 1.3 | 0.3 | 4.4 |
| LIP1-WT | | | | | | | | 1% SO/1% triton | 25.3 | 24.9 | 1.0 |
| | | | | | | | | 1% soy oil | 6.0 | 6.4 | 0.9 |
| | | | | | | | | 1% soy oil | 6.8 | 6.8 | 1.0 |
| | | | | | | | | 50% soy oil | 9.2 | 17.0 | 0.5 |
| | | | | | | | | 1% soy oil | 13.4 | 18.3 | 0.7 |
| | | | | | | | | 50% soy oil | 46.7 | 62.7 | 0.7 |

FIG. 2 is a graphical presentation of the results of table 8. The extent of palmitic acid release is plotted against the degree of hydrolysis. For each variant, only the point with highest degree of hydrolysis from table 7 were used for this graph (except for the wild type). When an improved variant is 100% specific for palmitic acid, then the DH versus palmitic acid release should follow line A. In case of a non-specific lipase this is line C. In case of 50% specificity this is line B. The results of the WT LIP1 all are below line C, indicating a preference for hydrolysing unsaturated fatty acid from (squares) soy oil. For the variants, all points (triangles) are between the B and C line, indicating an improved specificity towards palmitic acid when compared with the wild type LIP1 lipase.

### Example 6

### Lipase Activity in EPA Ethyl Ester Concentrate

The activity of the LIP1 variants with mutation G414V or G414T, were compared with the activity of wild type LIP1 lipase in an application type incubation on EPA ethyl ester concentrate (EPA-EE, Ocean Nutrition lot TS00010139, see table 9 for the composition). 245 μL of substrate (1% EPA-EE in 50 mM acetate buffer pH 4.5 with 3% triton X-100) was mixed with 70 μL enzyme sample of LIP1 mutants G414V or G414T and wild type LIP1. After 18 hours of incubation in a water bath at 37° C. under stirring, the reaction was stopped by adding 50 μL 1 M HCl. The fatty acids released were analyzed after extraction of the samples with chloroform using FAME according to the method disclosed above.

TABLE 9
Fatty acid composition of EPA ethyl ester concentrate.

| type of fatty acid | μmol/g | mol % |
|---|---|---|
| C12:0 | 0.41 | 0.01 |
| C13:0 | 0.44 | 0.01 |
| C14:0 | 4.2 | 0.14 |
| C16:0 | 19.8 | 0.67 |
| C16:1 | 15.1 | 0.51 |
| C16:2 | 2.1 | 0.07 |
| C16:3 | 2.0 | 0.07 |
| C16:4 | 4.9 | 0.17 |
| C18:0 | 134 | 4.5 |
| C18:1 | 294 | 9.9 |
| C18:2 | 53.9 | 1.8 |
| C18:3 | 23.5 | 0.79 |
| C18:4 | 74.9 | 2.53 |
| C20:0 | 16.0 | 0.54 |
| C20:1 | 93.5 | 3.2 |
| C20:2 | 26.6 | 0.90 |
| C20:3 | 20.4 | 0.69 |
| C20:4 | 179 | 6.03 |
| C20:5 | 1968 | 66.4 |
| C21:5 | 12.5 | 0.42 |
| C22:5 | 2.89 | 0.10 |
| C22:6 | 16.26 | 0.55 |

The data in table 10 show that incubation of EPA-EE substrate with variant G414V or variant G414T resulted in a reduced release of EPA fatty acids compared to the wild type LIP1. For variant G414V, this improved specificity towards non-EPA fatty acids allows to enrich the EPA-EE concentrate up to 77.3 mol % at a degree of hydrolysis around 30% with loss of EPA of 16%. This is a clear improvement compared with the performance of the LIP1-WT lipase. For the WT enzyme, at a same degree of hydrolysis, this enrichment was limited to an EPA content of 72.9% with accompanying loss of 24.1%.

TABLE 10
Effect of enzyme treatment on EPA ethyl ester concentrate

| | | EPA | Increase EPA | Degree of | EPA |
|---|---|---|---|---|---|
| | | content | content | hydrolysis | loss |
| variant | mutation | (%) | (%) | (%) | (%) |
| blank | — | 66.4 | 0.0 | 0.0 | 0.0 |
| LIP1-WT | — | 72.9 | 6.5 | 30.9 | 24.1 |
| CRL_061 | G414V | 77.3 | 10.9 | 27.8 | 16.0 |
| CRL_060 | G414T | 73.0 | 6.6 | 16.2 | 7.97 |

1. A polypeptide having lipase activity wherein the polypeptide is selected from the group consisting of
a) a polypeptide, which, when aligned with the polypeptide according to SEQ ID NO: 1, comprises at least the amino acid substitution G414X, wherein the numbering of amino acid position(s) is/are defined with reference to SEQ ID NO: 1, or corresponding position;
b) a polypeptide according to a), wherein the polypeptide has at least 80%, 85%, 90%, 91%, 92%, 93%, 94%, 95%, 96%, 97%, 98%% or 99% identity to the amino sequence of SEQ ID NO: 1;
c) a polypeptide encoded by a nucleic acid which has at least 80%, 85%, 90%, 91%, 92%, 93%, 94%, 95%, 96%, 97%, 98%, or 99% identity to the nucleotide sequence of SEQ ID NO: 2, wherein SEQ ID NO: 2 comprises at least one mutation resulting in at least the amino acid substitution G414X of a polypeptide according to SEQ ID NO: 1 or corresponding position, wherein the numbering of amino acid position(s) is/are defined with reference to SEQ ID NO: 1; and,
d) a polypeptide encoded by a nucleic acid comprising a sequence that hybridizes under low, medium and/or high stringency conditions to the complementary strand of sequence of SEQ ID NO: 2, wherein SEQ ID NO: 2 comprises at least one mutation resulting in at least the amino acid substitution G414X of a polypeptide according to SEQ ID NO: 1 or corresponding position, wherein the numbering of amino acid position(s) is/are defined with reference to SEQ ID NO: 1.

2. A polypeptide according to claim 1, wherein X represents an amino acid chosen from A, S, T and V.

3. A polypeptide according to claim 1, further comprising one or more amino acid substitutions chosen from the group consisting of I100V, S450A, L413M, L410F, S365A, S365Q, Y361W, F362L, and V409A.

4. A polypeptide according to claim 1, that is an isolated, substantially pure, pure, recombinant, synthetic or variant polypeptide of the polypeptide according to any of the preceding claims.

5. A polypeptide according to claim 1, wherein the polypeptide has a higher specificity towards myristate, palmitate and stearate relative to the specificity towards eicosapentanoate (EPA) than the specificity towards myristate, palmitate and stearate relative to the specificity towards eicosapentanoate of a corresponding wild-type polypeptide and/or wherein the polypeptide has a higher specificity towards palmitate relative to the specificity towards oleate and/or linoleate than the specificity towards palmitate relative to the specificity towards oleate and/or linoleate of a corresponding wildtype polypeptide.

6. A composition comprising a polypeptide according to claim 1.

7. A nucleic acid encoding a polypeptide according to claim 1.

8. A nucleic acid according to claim 7, wherein the nucleic acid has at least 80%, 85%, 90%, 95%, 96%, 97%, 98%, or 99% sequence identity to SEQ ID NO: 2, wherein SEQ ID NO: 2 comprises at least one mutation resulting in the amino acid substitution G414X, and optionally one or more amino acid substitutions chosen from the group consisting of I100V, S450A, L413M, L410F, S365A, S365Q, Y361W, F362L, and V409A, of a polypeptide according to SEQ ID NO: 1, wherein the numbering of amino acid position(s) is/are defined with reference to SEQ ID NO: 1.

9. An expression vector comprising a nucleic acid according to claim 7, operably linked to at least one control sequence that directs expression of the polypeptide in a host cell.

10. A recombinant host cell comprising a nucleic acid according to claim 7, or an expression vector according to claim 9.

11. A method for preparing a polypeptide according to claim 1, comprising cultivating a host cell according to claim 10 in a suitable fermentation medium, under conditions that allow expression of the polypeptide, and optionally recovering the polypeptide.

12. A process for preparing a product comprising an oil or fat comprising bringing an intermediary form of the product comprising oil or fat into contact with a polypeptide according to claim 1, or a composition according to claim 4, and preparing the product.

13. A process according to claim 12, wherein the product is a food or feed product.

14. A process according to claim 12, wherein the process further comprises separating a fatty acid.

15. Use of a polypeptide according to claim 1 to lower saturated fatty acids or monounsaturated fatty acids in an oil or fat, preferably wherein the oil is fish oil, soy oil, sunflower oil safflower oil, grapeseed oil, flaxseed oil or walnut oil.

16. Use according to claim 15, wherein the fatty acids are chosen from the group consisting of lauric acid (C12:0), myristic acid (C14:0) myristoleic acid (C14:1), palmitic acid (C16:0), palmitoleic acid (C16:1), stearic acid (C18:0), oleic acid (C18:1), arachidic acid (C20:0), 11-eicosenoic acid or gondoic acid (C20:1), docosanoic acid (C22:0) and erucic acid or brassidic acid (C22:1).
